SEQUENCE LISTING (6 sequences)

SEQ ID NO: 1 - DNA, 594 bp, Artificial Sequence
  CMV WT
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccg

SEQ ID NO: 2 - DNA, 591 bp, Artificial Sequence
  CMV-TrpO v1
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgttgaaaacagaactagttaactagtacgttgtattataagtaaggactagt

SEQ ID NO: 3 - DNA, 583 bp, Artificial Sequence
  CMV-TrpO v2
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagaactagttaactagtacgtcagatcgcctggagacgccatccacgctgttttgacctccatagaagacaccgactctac

SEQ ID NO: 4 - DNA, 591 bp, Artificial Sequence
  CMV-CuO v1
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgttgaaaacaaacagacaatctggtctgtttgtattataagtaaggactagt

SEQ ID NO: 5 - DNA, 630 bp, Artificial Sequence
  CMV-CuO v2
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgttgaaaacaaacagacaatctggtctgtttgtattataagtaaggactagtccatagaagacaccgactctactagaggatcgctagcgctaccggactcagatctcgagctcaagcttc

SEQ ID NO: 6 - DNA, 630 bp, Artificial Sequence
  CMV-CuO v3
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgaacaaacagacaatctggtctgtttgtacgccatccacgctgttttgacctccatagaagacaccgactctactagaggatcgctagcgctaccggactcagatctcgagctcaagcttc

(19) United States
(12) Patent Application Publication
(10) Pub. No.: US 2025/0277232 A1
(43) Pub. Date: Sep. 4, 2025

(54) LENTIVIRUS VECTOR WITH CONTROLLABLE EXPRESSION OF GENE OF INTEREST, AND PACKAGING METHOD THEREFOR

(71) Applicant: OBIO TECHNOLOGY (SHANGHAI) CORP., LTD., Shanghai (CN)

(72) Inventors: Jiali YANG, Shanghai (CN); Xinglin YANG, Shanghai (CN); Qingrui YOU, Shanghai (CN); Peimin MA, Shanghai (CN); Guodong JIA, Shanghai (CN)

(21) Appl. No.: 18/265,820

(22) PCT Filed: Dec. 6, 2021
(86) PCT No.: PCT/CN2021/135790

§ 371 Date: Jun. 7, 2023

(30) Foreign Application Priority Data
Dec. 7, 2020 (CN) 202011418514.0

(51) Int. Cl. C12N 15/86; C12N 7/00

(52) U.S. Cl. CPC C12N 15/86; C12N 7/00; C12N 2740/16043; C12N 2740/16052; C12N 2740/16061; C12N 2830/005

(57) ABSTRACT

The present application relates to a lentivirus vector with controllable expression of a gene of interest, and a packaging method therefor. The lentivirus packaging vector contains a first LTR, a reversely inserted gene expression cassette and a second LTR, wherein the first LTR is positioned upstream of the reversely inserted gene expression cassette in the direction of viral genome expression; the second LTR is positioned downstream of the reversely inserted gene expression cassette in the direction of viral genome expression; the gene expression cassette contains a promoter, a repressible operon and an optional gene of interest, which are connected in sequence; and in the presence of a repressor, the repressible operon is capable of repressing the expression of the gene of interest positioned downstream thereof.

### CROSS-REFERENCE TO RELATED APPLICATIONS

This application claims priority of Chinese Patent Application No. 202011418514.0, filed with the Chinese Patent Office on Dec. 7, 2020, entitled “Lentivirus packaging method for exogenous gene controllable expression”, the content of which is hereby incorporated by reference in its entirety.

### TECHNICAL FIELD

The present disclosure relates to the field of molecular biology, in particular, to a lentiviral vector for controllable expression of exogenous genes and a method for packaging the same.

### BACKGROUND

Viral vectors can bring genetic material into cells via the principle of the molecular mechanism that viruses bind to cell surface receptors with a high affinity and transmit their genomes into certain types of cells during infection. Viral vectors are widely used in basic research, gene therapy or vaccines.

Lentiviral vectors are vectors for gene therapy developed based on human immunodeficiency virus type I (HIV-1). Unlike general retroviral vectors, lentiviral vectors are capable of infecting both dividing cells and non-dividing cells.

Lentiviral vectors have been developed rapidly and are very deeply studied. By using the lentiviral vector, an exogenous gene can be effectively integrated into a chromosome of a host, thus achieving persistent expression. In terms of infection efficacy, the lentiviral vector can effectively infect various types of cells such as neuronal cells, hepatocytes, cardiomyocytes, tumor cells, endothelial cells, stem cells and the like, thereby achieving good effects on gene therapy. Gene therapy by using lentiviral vectors has been clinically studied in the United States with very satisfactory effects achieved, and therefore the lentiviral vector has a broad application prospect.

Current studies of viral vectors focus on aspects of infectivity, specificity, packaging capacity or the like, while little attention has been paid to the effects of exogenous genes packaged (i.e., gene of interest, GOI) on the virus. In practice, a considerable proportion of exogenous genes result in not being able to obtain effective viral particles. The reasons include the following. On the one hand, in the viral packaging process, an exogenous gene is usually driven by a strong promoter, and its expression has unpredictable effects on the condition of cells, the packaging, release, and other processes of viruses. For example, a long terminal repeat (LTR) of the HIV-1 genome includes two NFκB binding sites and the transcription of the HIV-1 genome is heavily dependent on activation of the classical NFκB pathway, whereas high expression of UBXN gene family members (such as UBXN1, N9 and N11) can block this pathway, thus greatly affecting the production of lentivirus. On the other hand, some genes, such as MARCH8, expressed on the envelope of lentivirus would not reduce the production of lentivirus but, upon overexpression, may severely reduce the infectivity of viral particles.

### SUMMARY

Various exemplary embodiments disclosed in the present disclosure provide a vector for lentiviral packaging, including a first long terminal repeat, a reversely inserted gene expression cassette, and a second long terminal repeat. The first long terminal repeat is positioned upstream of the reversely inserted gene expression cassette in a direction of viral genome expression. The second long terminal repeat is positioned downstream of the reversely inserted gene expression cassette in a direction of viral genome expression. The reversely inserted gene expression cassette includes a promoter, a repressible operon and an optional gene of interest which are linked in sequence. The repressible operon is capable of repressing expression of the gene of interest downstream of the repressible operon in the presence of a repressor.

According to another aspect, the present disclosure relates to a lentiviral vector packaging system, including the vector for lentiviral packaging as described above. The lentiviral vector packaging system is capable of producing HIV vector particles having only primary infectivity and no replication capacity.

According to yet another aspect, the present disclosure relates to a method for packaging a lentivirus, including transferring the lentiviral vector packaging system as described above into a host cell, and performing packaging in the presence of a repressor.

Compared to the relative art, the present disclosure has the following beneficial effects.

There is no need to determine whether the exogenous gene (i.e., the gene of interest) generates an effect on the lentivirus. In the absence of a repressor, the repression system would have no function and thus no effect on the expression of the gene of interest. In the case where significantly lower viral release than an average level is observed, in cooperation with a helper plasmid capable of expressing a repressor protein, the process of viral packaging can be carried out with the expression of exogenous gene inhibited, thereby eliminating the influence of the exogenous gene and increasing the yield of the specific virus without reconstructing a new shuttle vector, and thus saving time, labor and material resources.

### BRIEF DESCRIPTION OF THE DRAWINGS

In order to illustrate the technical solutions clearly in the specific embodiments or some techniques of the present disclosure, the accompanying drawings needed to be used in the description of specific embodiments or some techniques are briefly described below. It is apparent that the accompanying drawings as described below show embodiments of the present disclosure, and other drawings can also be obtained based on these drawings by those of ordinary skill in the art without creative work.

FIGS. 1 to 5 show the plasmid maps of vectors pcDNA3.1-CymR, CMV-CuO, EF1a-CuO, SFH-CuO and CAG-CuO, respectively, in an embodiment of the present disclosure.

FIG. 6 shows the results of the verification of repression efficiency of CymR-CuO in a lentiviral vector in an embodiment of the present disclosure.

FIG. 7 shows the plasmid map of CMV-TrpO in an embodiment of the present disclosure.

FIG. 8 shows the results of the verification of repression efficiency of tryptophan operon system in a lentiviral vector in an embodiment of the present disclosure.

FIG. 9 shows the plasmid map of EF1a-ToxO in an embodiment of the present disclosure.

FIG. 10 shows the results of the verification of repression efficiency of diphtheria toxin repressor regulatory system in a lentiviral vector in an embodiment of the present disclosure.

FIG. 11 shows the plasmid map of SFH-LacO in an embodiment of the present disclosure.

FIG. 12 shows the result of the verification of repression efficiency of lactose operon system in a lentiviral vector in an embodiment of the present disclosure.

FIG. 13 is a schematic diagram showing insertion sites of TrpO element relative to TATA Box of CMV in an embodiment of the present disclosure.

FIG. 14 shows the influence of different insertion sites of TrpO element on repression efficiency in an embodiment of the present disclosure.

FIG. 15 is a schematic diagram showing insertion sites of CuO element relative to TATA Box of CMV in an embodiment of the present disclosure.

FIG. 16 shows the influence of different insertion sites of CuO element on repression efficiency in an embodiment of the present disclosure.

FIG. 17 shows images showing the results for fluorescence observation under expression of C1V1(t/t)-TS-mCherry gene by a lentiviral vector in an embodiment of the present disclosure.

FIG. 18 shows the results of the titer test under expression of C1V1(t/t)-TS-mCherry gene by a lentiviral vector in an embodiment of the present disclosure.

FIGS. 19 to 21 show the plasmid maps of pSLenti-CMV-C1V1(t-t)-TS-mCherry-3xFLAG-PGK-puro-WPRE, pSLenti-CMV-CuO-C1V1(t-t)-TS-mCherry-3xFLAG-PGK-puro-WPRE, and pSLenti-CMV-TrpO-C1V1(t-t)-TS-mCherry-3xFLAG-PGK-puro-WPRE, respectively, in an embodiment of the present disclosure.

FIG. 22 shows the results of the virus titer test under expression of MCP-P65-HSF1 gene by a lentiviral vector in an embodiment of the present disclosure.

FIGS. 23 to 25 show the plasmid maps of pSLenti-CMV-MCP-P65-HSF1-2A-Hygro-WPRE, pSLenti-CMV-CuO-MCP-P65-HSF1-2A-Hygro-WPRE, and pSLenti-CMV-TrpO-MCP-P65-HSF1-2A-Hygro-WPRE, respectively, in an embodiment of the present disclosure.

FIG. 26 shows images showing the results of fluorescence and light field observation during packaging under the expression of CASP2 gene by a lentiviral vector in an embodiment of the present disclosure.

FIG. 27 shows the results of the virus titer test under the expression of CASP2 gene by a lentiviral vector in an embodiment of the present disclosure.

FIGS. 28 to 30 show plasmid maps of pSLenti-EF1-EGFP-P2A-Neo-CMV-CASP2-3xFLAG-WPRE, pSLenti-EF1-EGFP-P2A-Neo-CMV-CuO-CASP2-3xFLAG-WPRE, and pSLenti-EF1-EGFP-P2A-Neo-CMV-TrpO-CASP2-3xFLAG-WPRE, respectively, in an embodiment of the present disclosure.

FIG. 31 shows schematic diagrams of A) lentiviral packaging with a reverse expression cassette encoding no repressible operon, B) lentiviral packaging with a reverse expression cassette encoding a tryptophan operon, and C) lentiviral packaging with a reverse expression cassette encoding a Cumate-CuO regulable system.

### DETAILED DESCRIPTION

Reference to embodiments of the present disclosure will be made in detail and one or more examples of which are described below. Each of the examples is provided by way of explanation and does not limit the present disclosure. In fact, it will be apparent to those skilled in the art that various modifications and variations can be made to the present disclosure without departing from the scope or spirit of the present disclosure. For example, features illustrated or described as a part of one embodiment are applicable to another embodiment to generate a still further embodiment.

Thus, it is intended that the present disclosure covers such modifications and variations falling within the scope of the appended claims and their equivalents. Other subjects, features and aspects of the present disclosure are disclosed in or are apparent from the following detailed description. It is understood by those skilled in the art that the present discussion is a description of exemplary embodiments only and is not intended to limit broader aspects of the present disclosure.

Unless otherwise defined, all technical and scientific terms used herein have the same meaning as commonly understood by those skilled in the technical field to which this present disclosure belongs. The terms used herein in the specification of the present disclosure are only for the purpose of describing specific embodiments and are not intended to limit the present disclosure. As used herein, the term “and/or” includes any or all combinations of one or more of the associated listed items.

The present disclosure relates to a vector for lentiviral packaging, including a first long terminal repeat, a reversely inserted gene expression cassette, and a second long terminal repeat. The first long terminal repeat is positioned upstream of the reversely inserted gene expression cassette in a direction of viral genome expression. The second long terminal repeat is positioned downstream of the reversely inserted gene expression cassette in a direction of viral genome expression. The reversely inserted gene expression cassette includes a promoter, a repressible operon and an optional gene of interest which are linked in sequence. The repressible operon is capable of repressing expression of the gene of interest downstream of the repressible operon in the presence of a repressor.

In the present disclosure, the expression of the gene of interest can be regulated through the repressible operon, thereby achieving the selective inhibition of the expression of exogenous gene in the cells for lentiviral packaging, avoiding the negative influence of the exogenous gene on the packaging and production of lentivirus, and improving the production efficiency and titer of virus. Meanwhile, the expression level of the exogenous gene in a target cell is not reduced, which will not affect the therapeutic effect or study of the function of exogenous gene.

Term “vector” in the present disclosure refers to a nucleic acid delivery vehicle into which a polynucleotide can be packaged. In the case where a vector enables the expression of the protein encoded by the inserted polynucleotide, the vector is called an expression vector. A vector can be introduced into a host cell by transformation, transduction, or transfection, so that the genetic material element carried by the vector can be expressed in the host cell. In an embodiment, the vector is a plasmid.

In some embodiments, a distance between a site where a TrpO element of a tryptophan operon is inserted into the gene expression cassette and a TATA BOX of the promoter is less than or equal to 18 nucleotides, such as 17, 16, 15, 14, 13, 12, 11, 10, 9, 8, 7, 6, 5, 4, 3, 2 or 1 nucleotide(s).

In some embodiments, a distance between a site where a CuO element of a Cumate-CuO regulable system is inserted into the gene expression cassette and a TATA BOX of the promoter is 40 to 50 nucleotides, such as 41, 42, 43, 44, 45, 46, 47, 48 or 49 nucleotides.

The distance between the site where the above-mentioned element of the repressible operator is inserted and the TATA BOX of promoter refers to the number of nucleotide(s) between the first nucleotide (exclusive) at the 5′ end of the element and the first nucleotide (exclusive) at the 3′ end of the TATA BOX.

It should be noted that the eight nucleotides (including the 8th nucleotide) following the TATA BOX are considered to belong to a core region of the promoter. In an embodiment, the distance between the site where the TrpO element is inserted and the TATA BOX of the promoter is greater than 8 nucleotides. In the case where the TrpO element has a sequence that is overlapped with said eight nucleotides, the TrpO element can be closer to the TATA BOX.

The “promoter” is a DNA sequence that directs the binding of RNA polymerase and thereby initiates RNA synthesis. As used in the present disclosure, the promoter allows expression in a wide variety of types of cells and tissues. The promoter may be a non-cell-specific promoter. Alternatively; the promoter may be a cell-specific promoter, a cell type-specific promoter, a cell lineage-specific promoter, or a tissue-specific promoter, which allows the expression in their respective species-restricted types of cells or tissues. In particular embodiments, it may be desirable to use expression control sequences specific to cells, cell types, cell lineages or tissues, to achieve the cell type-specific, cell lineage-specific or tissue-specific expression of a desired polynucleotide sequence, e.g., expression of a nucleic acid encoding a polypeptide only in a subgroup of cell types, cell lineages, or tissues, or at specific developmental stages.

Exemplary examples of tissue-specific promoters include but are not limited to, B29 promoter (expressed in B cells), runt transcription factor (CBFa2) promoter (specifically expressed in stem cells), CD14 promoter (expressed in monocytes), CD43 promoter (expressed in leukocytes and platelets), CD45 promoter (expressed in hematopoietic cells), CD68 promoter (expressed in macrophages), CYP4503A4 or ALB promoter (expressed in hepatocytes), desmin promoter (expressed in muscle cells), elastase 1 promoter (expressed in pancreatic acinar cells), endothelial glycoprotein promoter (expressed in endothelial cells), fibroblast-specific protein 1 (FSP1) promoter (expressed in fibroblasts), fibronectin promoter (expressed in fibroblasts), fms-associated tyrosine kinase 1 (FLT1) promoter (expressed in endothelial cells), glial fibrillary acidic protein (GFAP) promoter (expressed in astrocytes), insulin promoter (expressed in pancreatic cells), integrin-α-2b (ITGA2B) promoter (megakaryocytes), intracellular adhesion molecule 2 (ICAM-2) promoter (endothelial cells), interferon-β (IFN-β) promoter (hematopoietic cells), keratin 5 promoter (expressed in keratinocytes), myoglobin (MB) promoter (expressed in muscle cells), myogenic differentiation 1 (MYOD1) promoter (expressed in muscle cells), nephropathy protein promoter (expressed in podocytes), bone γ-carboxyl glutamic acid protein 2 (OG-2) promoter (expressed in osteoblasts), 3-ketoacid CoA transferase 2B (Oxct2B) promoter (expressed in haploid spermatocytes), surface activating protein B (SP-B) promoter (lung cell), synapsin promoter (expressed in neuronal cells), or Wiskott-Aldrich syndrome protein (WASP) promoter (expressed in hematopoietic cells).

In some embodiments, the promoter is a non-cell-specific promoter. Exemplary non-cell-specific promoters include but are not limited to, cytomegalovirus (CMV) immediate-early promoter, viral simian virus 40 (SV40) (e.g., early or late) promoter, Moloney murine leukemia virus (MoMLV) LTR promoter, Rous sarcoma virus (RSV) LTR promoter, herpes simplex virus (HSV) (thymidine kinase) promoter, cowpox virus H5, P7.5 and P11 promoters, elongation factor 1-alpha (EF1a) promoter, early growth response 1 (EGR1) promoter, ferritin H (FerH) promoter, ferritin L (FerL) promoter, glyceraldehyde-3-phosphate dehydrogenase (GAPDH) promoter, eukaryotic translation initiation factor 4A1 (EIF4A1) promoter, heat shock 70 kDa protein 5 (HSPA5) promoter, heat shock protein 90 kDa-β member 1 (HSP90B1) promoter, heat shock protein 70 kDa (HSP70) promoter, β-kinesin (β-KIN) promoter, human ROSA26 locus, ubiquitin C promoter (UBC), phosphoglycerate kinase-1 (PGK) promoter, cytomegalovirus enhancer/chicken β-actin (CAG) promoter, or β-actin promoter. The non-cell-specific promoters enable better versatility and expression efficiency of the described gene expression cassettes.

In some embodiments, the promoter is CMV, EF1a, SFH, CAG, CBh, UBC, SFFV, SV40, RSV, mCMV, GAPDH, PGK, CASI, SMVP, GUSB (hGBp) or UCOE promoters.

In some embodiments, the gene of interest is inserted downstream of the repressible operon. The repressible operon exerts a negative effect on viral packaging in the case where the gene of interest is expressed.

In the present disclosure, “a negative effect on viral packaging” refers to reducing the packaging efficiency of the virus, slowing or hindering the release of lentivirus, or directly killing the cells for lentiviral packaging. Moreover, the gene of interest would take up excessive enzymes and resources related to RNA transcription and protein translation in cells if it is possible to be transcribed and translated in large quantities, thereby indirectly inhibiting the transcription and translation of the functional genes of the recombinant virus. The effects in these two aspects reduce the production efficiency of recombinant virus in the cells for lentiviral packaging, resulting in an insufficient virus titer.

In some embodiments, the gene of interest has cytotoxicity to cells for lentiviral packaging.

In some embodiments, the gene of interest is selected from a suicide gene, an apoptotic gene (or programmed cell death gene), or an oncogene.

Common suicide gene systems may include tk-GCV system, CD-5-FC system, gpt-6-TX system, P450 2BI-CPA system, and the like.

In the present disclosure, the “apoptotic gene” may be used interchangeably with “programmed cell death gene”, including but not limited to, Bcl-2 gene, P53 gene, cytochrome C gene, apoptotic protease activating factor 1 (Apaf-1) gene, genes of Caspase family proteins, and the like.

In an embodiment, the Caspase family proteins may be categorized as initiator caspases (such as Caspase 8, Caspase 9, or Caspase 10) and effector caspases (such as Caspase 3, Caspase 6, or Caspase 7).

The main types of oncogenes include tyrosine kinases (such as src), other protein kinases (such as raf), G proteins (such as ras), growth factors (such as Sis), growth factor receptors (such as ErbB), and proteins within nucleus (such as transcription factor MYC).

In some embodiments, the gene of interest is a cell cycle-associated gene. For example, the gene of interest is a gene of Caspase family protein and a MAPK signaling pathway-related gene.

In some embodiments, the gene of interest directly inhibits replication and/or assembly of virus.

In some embodiments, the gene of interest is selected from C1V1(t/t)-TS-mCherry fusion gene, MCP-P65-HSF1 fusion gene, UBXN gene family, MARCH8 gene, or M2BP gene.

In some embodiments, the UBXN gene family is selected from UBXN1, N9 or N11.

In some embodiments, the vector for lentiviral packaging further includes at least one of a reporter gene, an enhancer, an internal ribosome entry site, or a terminator.

According to another aspect, the present disclosure relates to a lentiviral vector packaging system, including the vector for lentiviral packaging as described above. The lentiviral vector packaging system can produce HIV vector particles having only primary infectivity and no replication capacity.

In some embodiments, the lentiviral vector packaging system is a two-plasmid packaging system, a three-plasmid packaging system, or a four-plasmid packaging system.

In some embodiments, the HIV vector particles are HIV-1 or HIV-2 vector particles.

According to yet another aspect, the present disclosure relates to a method for packaging a lentivirus, including:
transferring the lentiviral vector packaging system as described above into a host cell, and performing packaging in the presence of a repressor.

In some embodiments, the host cell is a mammalian cell or an avian animal cell.

In some embodiments, the host cell can also be a fish cell or an amphibian cell.

In some embodiments, the host cell is a rodent cell, such as rat, mouse, or hamster.

In some embodiments, the host cell is a primate cell. In an embodiment, the host cell is a human cell.

In some embodiments, the host cell is a primary cell, such as a tumor cell, a liver cell, a cardiomyocyte, a neuron, an endothelial cell, a stem cell, and the like.

In some embodiments, the host cell is a cell line.

Common cell lines are as follows:
human-derived cell lines:
293, IMR-90, W1-38, A549, A431, BHL-100, BeWo, Caco-2, Chang, HCT-15, HeLa, HEp-G2, HEp-2, HT-1080, HT-29, JEG-2, MCF7, KB, Saos-2, WI-38, WISH, WS1, HUVEC, EB-3, Raji, IM-9, Daudi, H9, HL-60, Jurkat, K-562, U937, and KG-1;
mouse-derived cell lines:
McCoy, BALB/3T3, 3T6, A9, AtT-20, Clone M-3, I-10, Y-1, WEHI-3b, ES-D3, and F9;
hamster-derived cell lines:
BHK-21, HaK, and CHO-K1;
rat-derived cell lines:
AR42J, BRL3A, Clone 9, H4--II-E-C3, GH1, GH3, IEC-6, L2, XC, LLC-WRC 256, Jensen, Rat2(TK-), and PC12, L6; and
cell lines derived from other animals:
D-17, BT, MARC-145, CV-1, COS-1, COS-3, COS-7, Vero, B95-8, and CRFK.

Embodiments of the present disclosure will be described in detail below in combination with examples.

### Example 1. Packaging of Lentivirus and Titer Test

### I. Packaging of Lentivirus

A method for viral packaging using lentiviral vectors specifically includes the follows.
1. One day before transfection, 293T cells were seeded onto a culture dish. One hour before transfection, the culture dish was taken out and, with the spent cell culture medium discarded and replaced with Opti-MEM medium, placed back in the incubator. Then, a complex of transfection reagent and plasmid was prepared following these steps.
2. Viral vector plasmids prepared for transfection (including backbone plasmids pCAG-gagpol, envelope protein plasmids pHCMV-VSVG and shuttle plasmids) were dissolved in Opti-MEM medium, gently mixed, and left standing to obtain a diluted solution of plasmid. The backbone plasmids were pCAG-gagpol, pCAG-gagpol-CMV-CymR or pCAG gagpol-CMV-TrpR. The envelope protein plasmids were pHCMV-VSVG, pHCMV-VSVG-CMV-CymR, or pHCMV-VSVG-CMV-TrpR.
3. The transfection reagent was dissolved in Opti-MEM medium, gently mixed and left standing to obtain a diluted solution of transfection reagent. The diluted solution of transfection reagent was added dropwise to the diluted solution of plasmid with gentle mixing during the addition, and then kept at room temperature for 15 to 25 mins, allowing the DNA and the transfection reagent to be thoroughly combined to form a stable transfection complex. To the taken culture plate, the prepared complex of DNA and transfection reagent was added. The culture plate was then put back into the incubator.
4. After 5 to 8 hours, the medium was aspirated and, after washing the dish with phosphate buffered saline (PBS), replaced with fresh complete medium for culturing. For the tryptophan operon group (i.e., pSLenti-TK PolyA-mCherry-TrpO-CMV-SFH-EGFP-P2A-Puro-WPRE), the complete medium containing 0.3 mM tryptophan was used in the replacement.
5. At 48 hours after transfection, the first virus harvest was performed by collecting the medium into a 50 ml centrifuge tube, and then the spent medium was replaced with fresh complete medium. At 72 hours after transfection, the second virus harvest was performed by collecting the medium into a 50 ml centrifuge tube, and then the cells were discarded.
6. The harvested supernatant was centrifuged at 3500 rpm at room temperature for mins and transferred to a fresh 50 ml centrifuge tube. After centrifugation at 30000 rpm at 4° C. for 2 hours in an ultracentrifuge, the supernatant was carefully discarded. The centrifuge tube was inverted and placed on sterilized blotting paper. The precipitate was resuspended in Dulbecco's phosphate-buffered saline (DPBS), then collected into a 1.5 ml EP tube, and stored in a refrigerator at −80° C.

### II. Lentivirus Titer Test

The method for testing lentivirus titer specifically includes determining lentivirus titer by Real-time quantitative PCR. The specific procedure is as follows.

A sample was prepared as follows. 293T cells were seeded in a 24-well plate at 1×10⁵ cells per well. The lentiviruses were added on day 2, and the medium was replaced with the fresh medium after 12 to 20 hours. At 72 hours after infection, photos were taken for recording the fluorescence. After that, the 293T cells were collected for extraction of genomic DNA, and the titer was tested through quantitative PCR.

The Real-time quantitative PCR was performed on ABI7500 instrument. Reagent SYBR Master Mixture used was from TAKARA.
1. The reaction system was prepared according to the following proportions:
   SYBR premix ex taq: 10 μl;

### ROX: 0.4 μl;

Forward primer (25 μM): 0.5 μl;
Reverse primer (25 μM): 0.5 μl;

### Genomic DNA: 2.0 μl; and

water: 6.6 μl.
2. A procedure of two-step Real-time quantification was performed, including pre-denaturation at 95° C. for 15 seconds; and denaturation at 95° C. for 5 seconds and annealing extension at 60° C. for 34 seconds in each cycle, with a total of 40 cycles. The absorbance value was read in each annealing extension stage.

The PCR procedure was as follows:

### Cycle 1: (1×)

Step 1: 95.0° C. for 15 seconds;

### Cycle 2: (40×)

Step 1: 95.0° C. for 5 seconds,
Step 2: 60.0° C. for 34 seconds; and
initiation of data collection and real-time analysis.
3. A melting curve was prepared. After PCR, denaturation was performed at 95° C. for 1 minute, followed by cooling down to 55° C. to allow fully binding of the DNA double strands. Then, the reaction temperature was increased from 55° C. to 95° C., with an increase of 0.5° C. per cycle and each step holding for 30 seconds, as the absorbance value was read.

The procedure for preparing a melting curve was as follows:

### Cycle 3: (1×)

Step 1: 95.0° C. for 1 minute;

### Cycle 4: (1×)

Step 1: 55.0° C. for 1 minute;

### Cycle 5: (81×)

Step 1: 55.0° C. to 95.0° C., each step holding for 30 seconds,
the set temperature was increased by 0.5° C. after every 2 cycles in Cycle 5.

### Example 2. Selection of Repressible Operon

A total of four transcriptional regulatory systems (i.e., CymR-CuO, tryptophan operon, diphtheria toxin repressor regulatory system, and lactose operon) were tested in the present disclosure.

I. Firstly, cis-acting elements of these four transcriptional regulatory systems were respectively introduced into the lentiviral vectors to test whether the expression of exogenous gene was inhibited in a eukaryotic system.

### 1. CymR-CuO System

The CymR-CuO system is a class of regulatable expression systems that function by the following principle.

CymR protein can specifically bind to CuO element in the absence of Cumate, thereby inhibiting the gene transcription. In the presence of Cumate, CymR bound to Cumate would detach from the CuO element, thus allowing normal expression of a gene.

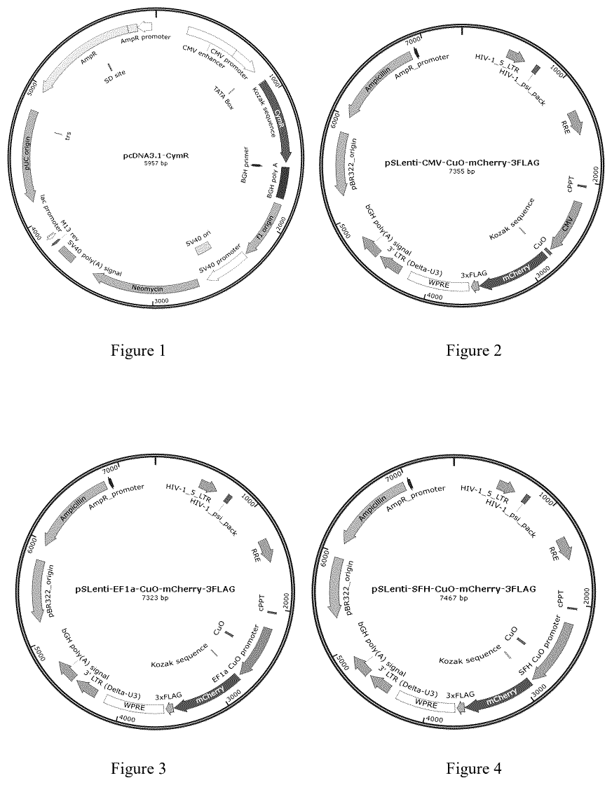

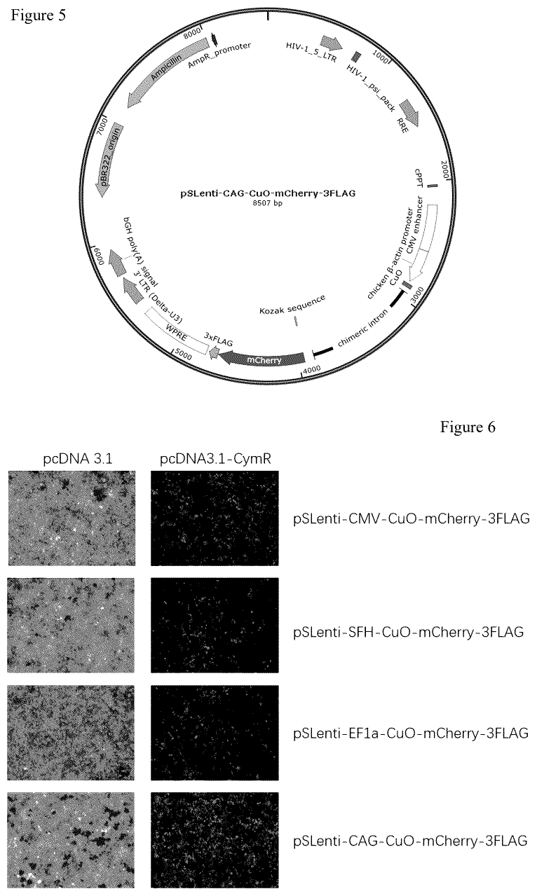

In the present disclosure, a series of promoter vectors CMV-CuO, EF1a-CuO, SFH-CuO and CAG-CuO were constructed. It was confirmed that the CuO element in cooperation with the CymR vector (pcDNA3.1-CymR) can effectively inhibit the gene expression. The plasmid map of pcDNA3.1-CymR is shown in FIG. 1. The plasmid maps of CMV-CuO, EF1a-CuO, SFH-CuO, and CAG-CuO promoter vectors are shown in FIGS. 2 to 5, respectively.

293T cells were co-transfected with the lentiviral vector encoding the CuO element and either pcDNA3.1 empty vector or pcDNA3.1-CymR. Fluorescence photographs were taken after 24 hours. It can be seen that the expression efficiency of CMV-CuO, EF1a-CuO, SFH-CuO and CAG-CuO promoters were significantly inhibited under the action of CymR protein (see FIG. 6).

### 2. Tryptophan Operon System

In the presence of tryptophan, the Trp repressor (TrpR) protein can specifically bind to the TrpO element and thus inhibits gene transcription. This tryptophan operon system was found in prokaryotic cells and had long been used as an explanatory demonstration of gene regulation. However, this tryptophan operon system has not been applied in eukaryotic cells because tryptophan is necessary for the culture of mammalian cells.

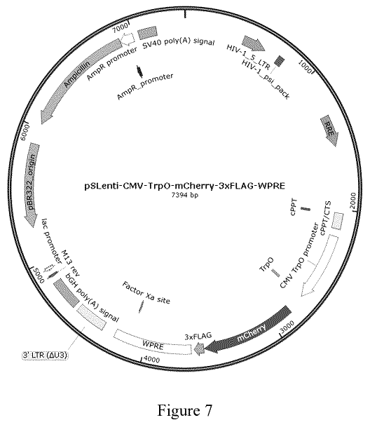

In the present disclosure, a series of promoter vectors such as CMV-TrpO, EF1a-TrpO, SFH-TrpO, CAG-TrpO were constructed. It was confirmed that the TrpO element in cooperation with the TrpR vector (pcDNA3.1-TrpR) can effectively inhibit the gene expression in the presence of tryptophan. The plasmid map of CMV-TrpO is shown in FIG. 7. The design of plasmid maps of EF1a-TrpO, SFH-TrpO, and CAG-TrpO promoter vectors can refer to FIGS. 2 to 5 on the basis of FIG. 7, which will not be repeated.

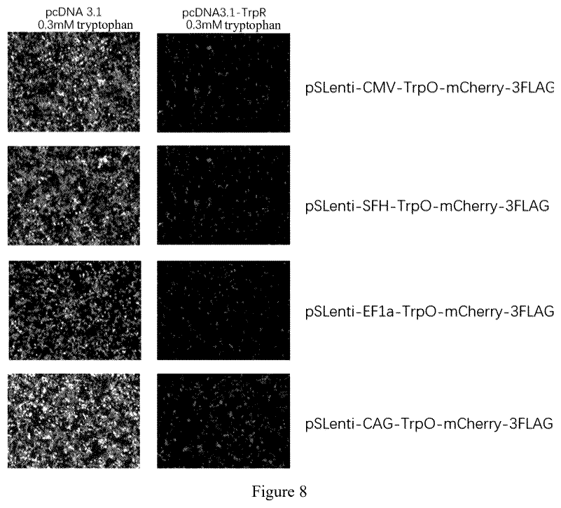

293T cells were co-transfected with the lentiviral vector encoding the TrpO element and either pcDNA3.1 empty vector or pcDNA3.1-TrpR, with 0.3 mM tryptophan added. Fluorescence photographs were taken after 24 hours. It can be seen that the expression efficiency of CMV-TrpO, EF1a-TrpO, SFH-TrpO and CAG-TrpO promoters were significantly inhibited under the action of TrpR protein (see FIG. 8).

### 3. Diphtheria Toxin Repressor Regulatory System

The diphtheria toxin repressor DtxR protein can specifically bind to the ToxO element and thus inhibits gene transcription. In the present disclosure, a series of promoter vectors such as CMV-ToxO, EF1a-ToxO, SFH-ToxO, CAG-ToxO were constructed. 293T cells were co-transfected with the lentiviral vector encoding the ToxO element and either pcDNA3.1 empty vector or pcDNA3.1-DtxR, with divalent iron ions added. As a result, the gene of interest was not found to be significantly inhibited (see FIG. 10).

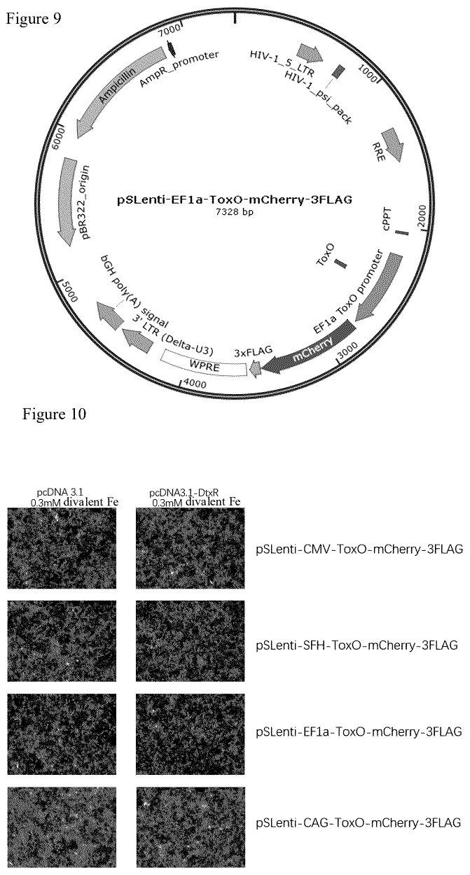

The plasmid map of EF1a-ToxO is shown in FIG. 9. The design of plasmid maps of CMV-ToxO, SFH-ToxO and CAG-ToxO promoter vectors can refer to FIGS. 2 to 5 on the basis of FIG. 9, which will not be repeated.

### 4. Lactose Operon System

The lactose operon repressor LacI protein can specifically bind to the LacO element and thus inhibits gene transcription. In the presence of isopropyl β-D-1-thiogalactopyranoside (IPTG), the LacI protein binds to the IPTG and is subjected to conformational change, thereby losing its binding ability and allowing the expression of downstream gene. The lactose operon system is widely used in prokaryotic cells for inducing the expression of exogenous genes.

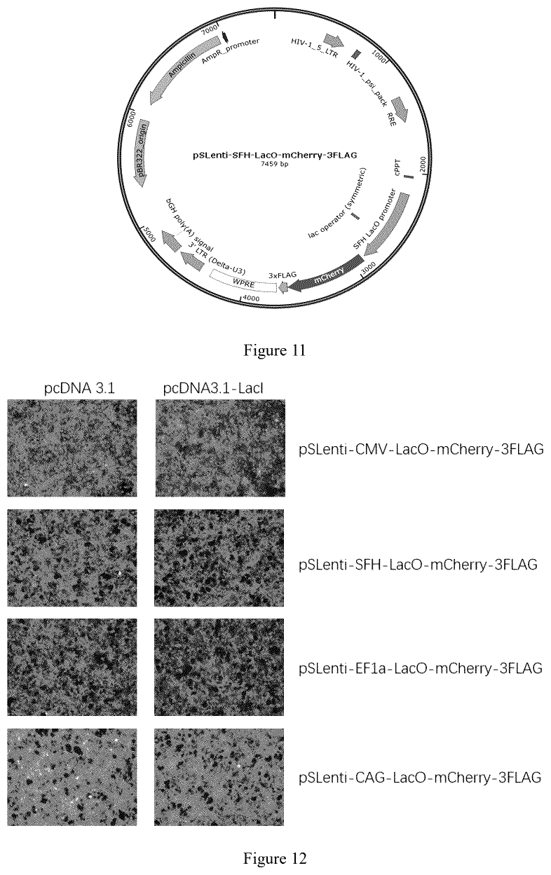

In the present disclosure, a series of promoter vectors such as CMV-LacO, EF1a-LacO, SFH-LacO, CAG-LacO were constructed. 293T cells were co-transfected with the lentiviral vector encoding the LacO element and either pcDNA3.1 empty vector or pcDNA3.1-LacI. As a result, the gene of interest was not found to be significantly inhibited (see FIG. 12).

The plasmid map of SFH-LacO is shown in FIG. 11. The design of plasmid maps of CMV-LacO, EF1a-LacO, and CAG-LacO promoter vectors can refer to FIGS. 2 to 5 on the basis of FIG. 11, which will not be repeated.

### II. Optimization of Insertion Sites of Repressible Operon Elements

### 1. TrpO Element

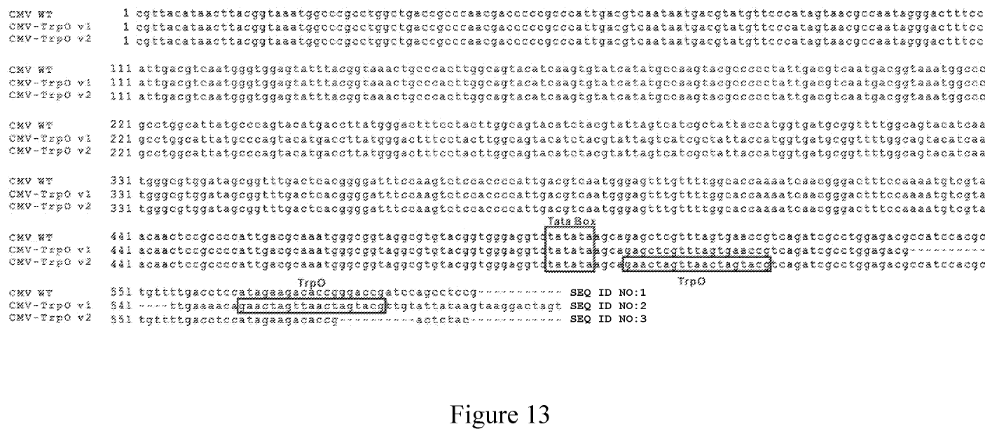

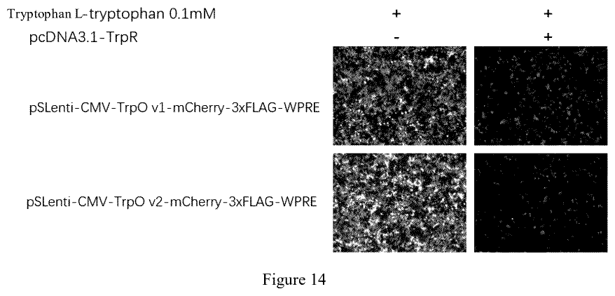

The distance between the TrpO element and the TATA Box of CMV can vary. The inventors have designed two promoters as shown in FIG. 13. At 48 hours after co-transfection with the plasmid encoding the TrpO element and pcDNA3.1-TrpR (with 0.1 mM tryptophan added), the fluorescence was observed. The experiment shows that varying the distance between the TrpO element and the TATA Box can result in different inhibitory effects (see FIG. 14). The TrpO v2 promoter sequence with a better inhibition effect was used in subsequent steps in the present disclosure.

### 2. CuO Element

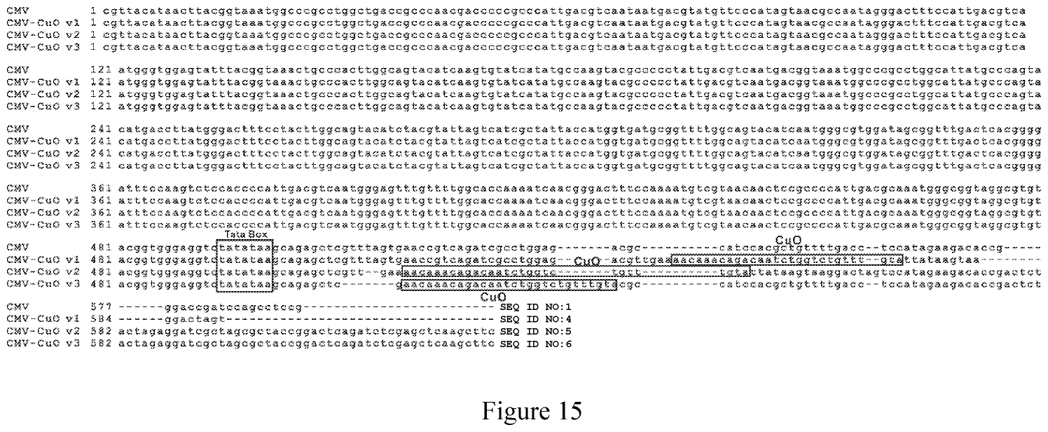

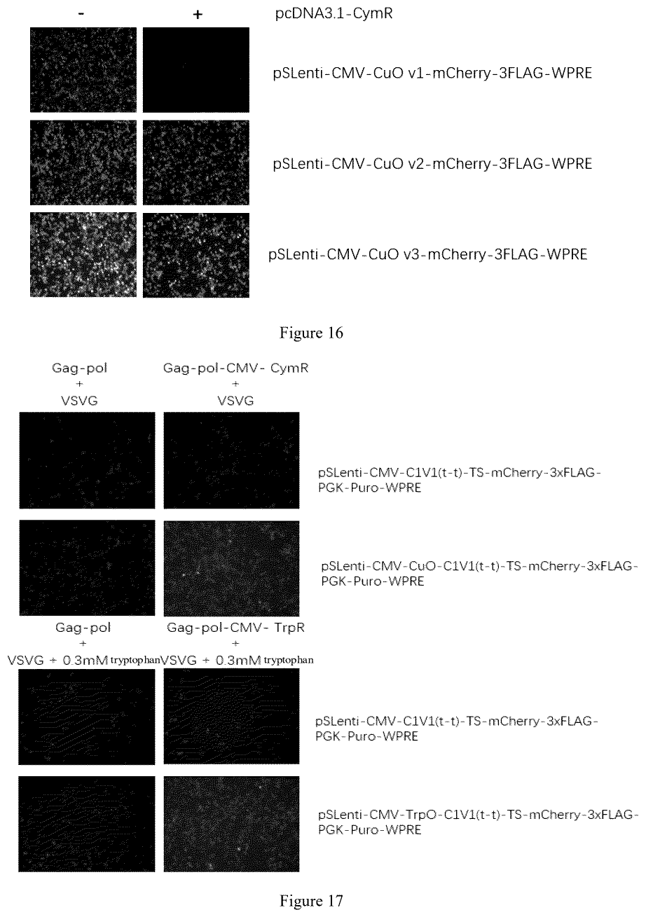

The distance between the CuO element and the TATA Box of CMV can vary. The inventors have designed three promoters, as shown in FIG. 15. At 48 hours after co-transfection with the plasmid encoding the CuO element and pcDNA3.1-CymR, the fluorescence was observed. The experiment shows that varying the distance between the CuO element and the TATA Box can result in different inhibitory effects (see FIG. 16). The CuO v1 promoter sequence with a better inhibition effect was used in subsequent steps in the present disclosure.

### Example 3 Lentiviral Vectors Expressing C1V1(t/t)-TS-mCherry Gene

The proteins that combine channelrhodopsin-1 with Volvox channelrhodopsin-1 (VChR1) are collectively referred to as C1V1. In the production of virus, it was found that lentiviral particles could not be packaged and obtained in the case where C1V1(t/t)-TS-mCherry was incorporated into a common lentiviral vector.

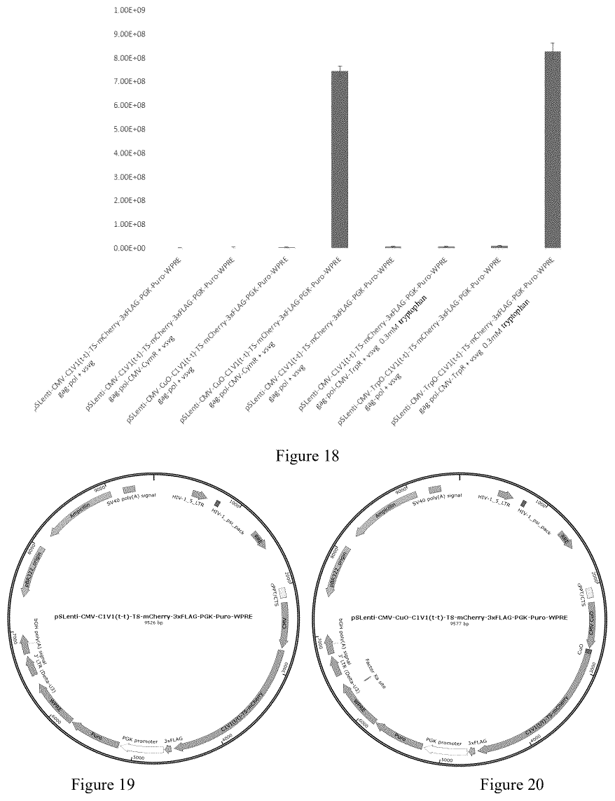

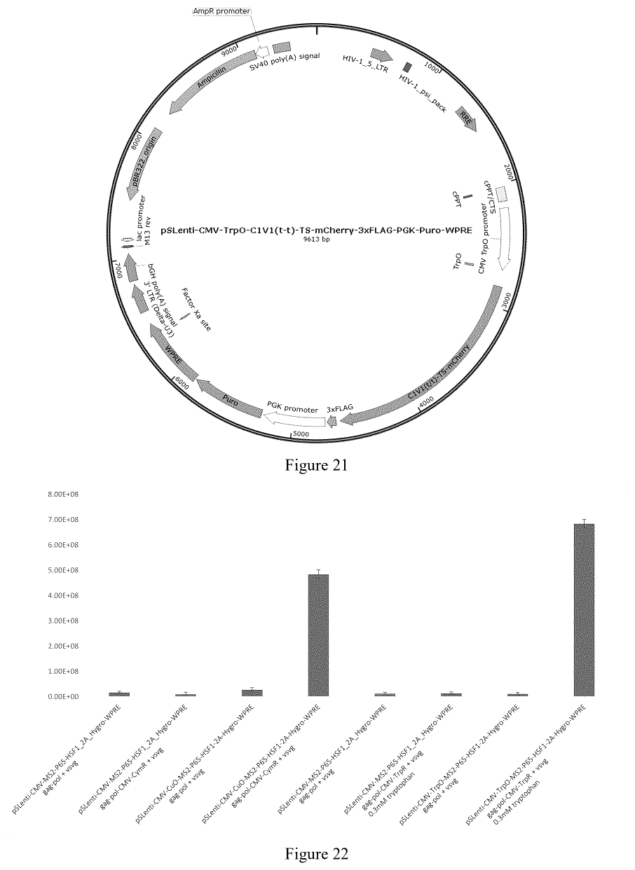

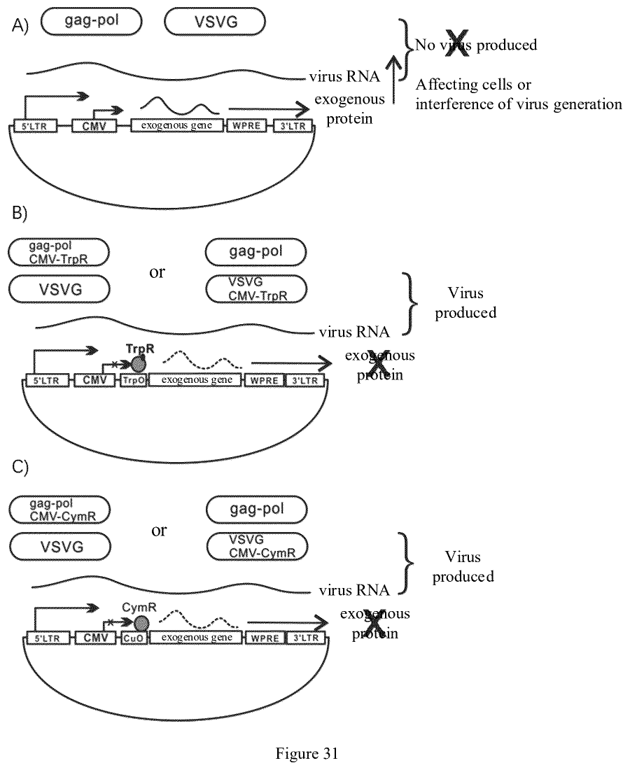

By using the lentiviral vector packaging system of the present disclosure and packaging in the presence of a repressor, the viral packaging titer can be effectively enhanced (see FIGS. 17 and 18). The plasmid maps of plasmids pSLenti-CMV-C1V1(t-t)-TS-mCherry-3xFLAG-PGK-puro-WPRE, pSLenti-CMV-CuO-C1V1(t-t)-TS-mCherry-3xFLAG-PGK-puro-WPRE, and pSLenti-CMV-TrpO-C1V1(t-t)-TS-mCherry-3xFLAG-PGK-puro-WPRE used in the example are shown in FIGS. 19, 20, and 21, respectively. The principle can refer to the illustration in FIG. 31.

### Example 4 Lentiviral Vectors Expressing MCP-P65-HSF1 Gene

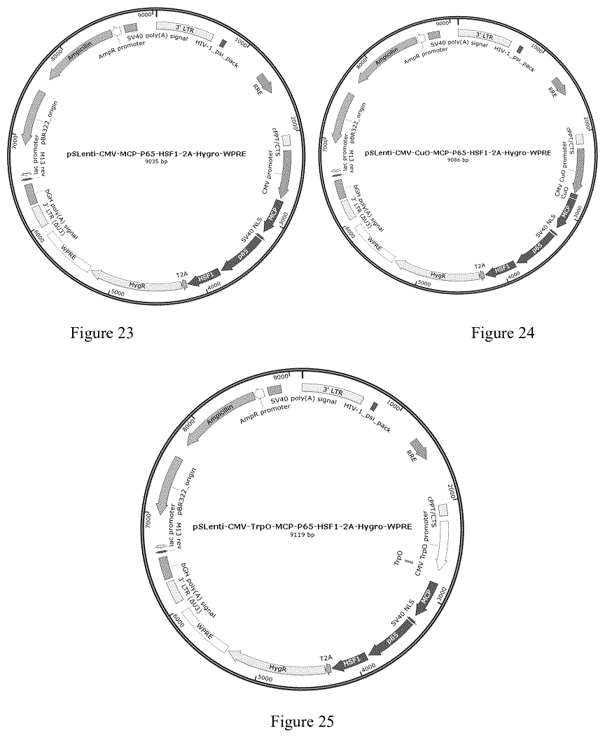

In the production of virus, it was found that the total amount of viral particles obtained was very low in the case where the MCP-P65-HSF1 gene was incorporated into a common lentiviral vector. By using CMV-CuO and CMV-TrpO in cooperation with CymR and TrpR respectively for viral packaging, the virus titers were increased by 28 to 50 folds (see FIG. 22). The plasmid maps of plasmids pSLenti-CMV-MCP-P65-HSF1-2A-Hygro-WPRE, pSLenti-CMV-CuO-MCP-P65-HSF1-2A-Hygro-WPRE, and pSLenti-CMV-TrpO-MCP-P65-HSF1-2A-Hygro-WPRE used in this example are shown in FIGS. 23, 24, and 25, respectively. The principle can refer to the illustration in FIG. 31.

### Example 5 Lentiviral Vectors Expressing CASP2 Gene

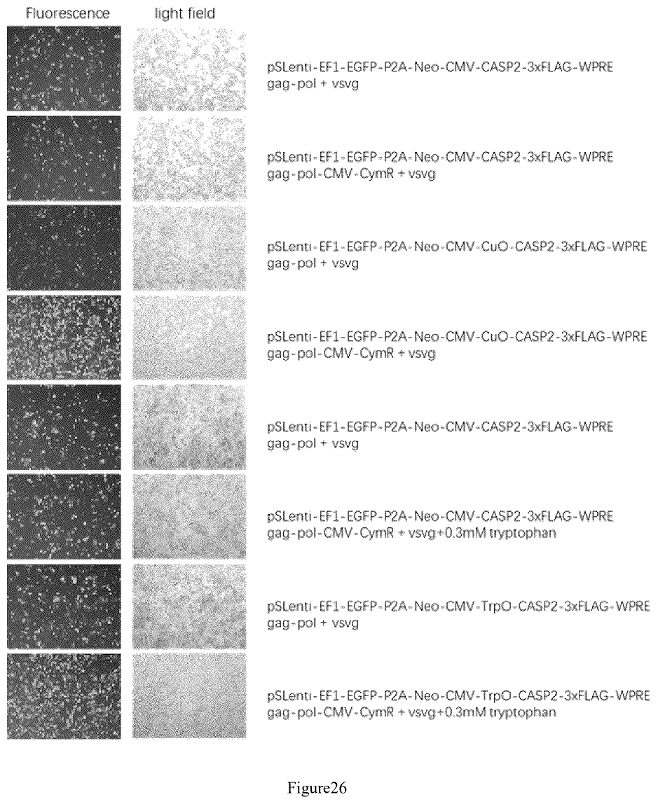

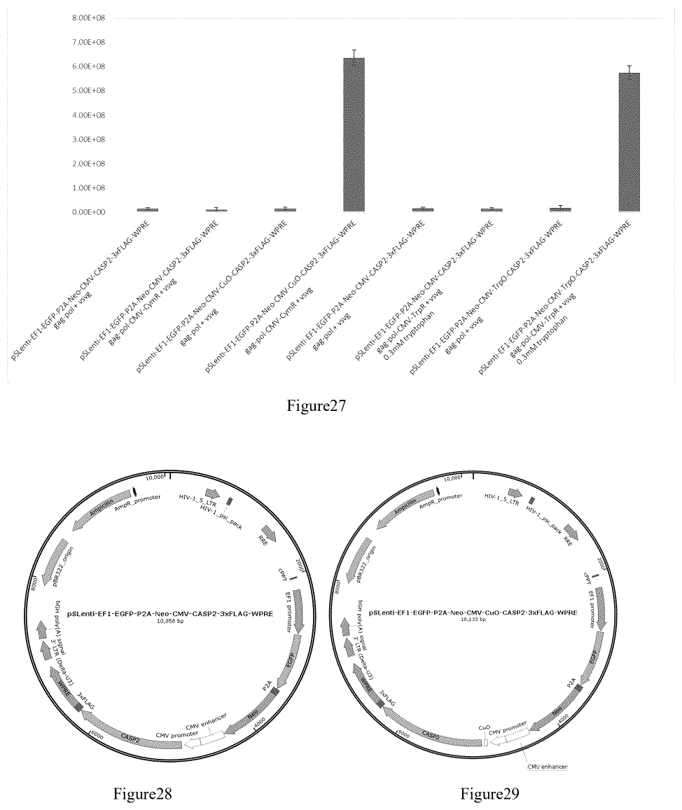

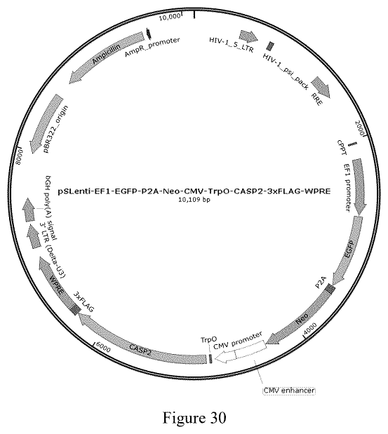

The expression of CASP2 gene can activate the apoptotic pathway of cells, thus in the case where the CASP2 gene was incorporated into a common lentiviral vector, a large number of cells were subjected to apoptosis during the packaging, thereby obtaining a very low total amount of viral particles (see FIG. 26). By using CMV-CuO and CMV-TrpO in cooperation with CymR and TrpR respectively for viral packaging, the virus titers were increased by 32 to 45 folds (see FIG. 27). The plasmid maps of plasmids pSLenti-EF1-EGFP-P2A-Neo-CMV-CASP2-3xFLAG-WPRE, pSLenti-EF1-EGFP-P2A-Neo-CMV-CuO-CASP2-3xFLAG-WPRE, and pSLenti-EF1-EGFP-P2A-Neo-CMV-TrpO-CASP2-3xFLAG-WPRE used in this example are shown in FIGS. 28, 29, and 30, respectively. The principle can refer to the illustration in FIG. 31.

The technical features of the examples described above can be combined arbitrarily. For the sake of conciseness, all possible combinations of the technical features of the above examples are not described. However, as long as these combinations of technical features do not contradict each other, they should be considered to be within the scope of this specification.

The examples described above in a specific and detailed manner merely express several embodiments of the present disclosure. However, they should not be construed as a limitation to the scope of the present disclosure. It should be noted that a number of variations and improvements can be made for a person of ordinary skill in the art without departing from the conception of the present disclosure, all of which fall within the protection scope of the present disclosure. Therefore, the protection scope of the present disclosure shall be subject to the appended claims.

1. A vector for lentiviral packaging, comprising a first long terminal repeat, a reversely inserted gene expression cassette, and a second long terminal repeat,
wherein the first long terminal repeat is positioned upstream of the reversely inserted gene expression cassette in a direction of viral genome expression,
the second long terminal repeat is positioned downstream of the reversely inserted gene expression cassette in a direction of viral genome expression,
the reversely inserted gene expression cassette comprises a promoter, a repressible operon and an optional gene of interest which are linked in sequence, and
the repressible operon is capable of repressing expression of the gene of interest downstream of the repressible operon in the presence of a repressor.

2. The vector for lentiviral packaging according to claim 1, wherein the repressible operon is selected from a tryptophan operon and/or a Cumate-CuO regulable system.

3. The vector for lentiviral packaging according to claim 2, wherein a distance between a site where a TrpO element of the tryptophan operon is inserted into the gene expression cassette and a TATA BOX of the promoter is less than or equal to 18 nucleotides.

4. The vector for lentiviral packaging according to claim 2, wherein a distance between a site where a CuO element of the Cumate-CuO regulable system is inserted into the gene expression cassette and a TATA BOX of the promoter is 40 to 50 nucleotides.

5. The vector for lentiviral packaging according to claim 1, wherein the promoter is CMV, EF1a, SFH, CAG, CBh, UBC, SFFV, SV40, RSV, mCMV, GAPDH, PGK, CASI, SMVP, GUSB or UCOE promoters.

6. The vector for lentiviral packaging according to claim 1, wherein a gene of interest is inserted downstream of the repressible operon, and the repressible operon exerts a negative effect on viral packaging in the case where the gene of interest is expressed.

7. The vector for lentiviral packaging according to claim 6, wherein the gene of interest has cytotoxicity to cells for lentiviral packaging.

8. The vector for lentiviral packaging according to claim 7, wherein the gene of interest is selected from a suicide gene, an apoptotic gene, or an oncogene.

9. The vector for lentiviral packaging according to claim 6, wherein the gene of interest is a cell cycle-associated gene.

10. The vector for lentiviral packaging according to claim 6, wherein the gene of interest directly inhibits replication and/or assembly of virus.

11. The vector for lentiviral packaging according to claim 6, wherein the gene of interest is selected from C1V1(t/t)-TS-mCherry fusion gene, MCP-P65-HSF1 fusion gene, UBXN gene family such as UBXN1, N9, and N11, MARCH8 gene, or M2BP gene.

12. The vector for lentiviral packaging according to claim 1, wherein the vector for lentiviral packaging further comprises at least one of a reporter gene, an enhancer, an internal ribosome entry site, or a terminator.

13. A lentiviral vector packaging system, comprising a lentiviral packaging vector of claim 1, wherein the lentiviral vector packaging system is capable of producing HIV vector particles having only primary infectivity and no replication capacity.

14. The lentiviral vector packaging system according to claim 13, wherein the lentiviral vector packaging system is a two-plasmid packaging system, a three-plasmid packaging system, or a four-plasmid packaging system.

15. A method for packaging a lentivirus, comprising:
transferring a lentiviral vector packaging system of claim 13 into a host cell, and performing packaging in the presence of a repressor.
